(19) United States
(12) Patent Application Publication
(10) Pub. No.: US 2004/0132111 A1
(43) Pub. Date: Jul. 8, 2004

(54) Assay for oxidative stress

(76) Inventors: Ali Banan, Gurnee, IL (US); Ali Keshavarzian, Evanston, IL (US)

Correspondence Address: WELSH & KATZ, LTD, 120 S RIVERSIDE PLAZA, 22ND FLOOR, CHICAGO, IL, 60606, US

(21) Appl. No.: 10/626,380

(22) Filed: Jul. 24, 2003

Related U.S. Application Data
(63) Continuation-in-part of application No. 10/263,207, filed on Oct. 2, 2002.

(51) Int. Cl. G01N 33/53; G01N 33/567

(52) U.S. Cl. 435/7.2

(57) ABSTRACT

The presence of oxidative stress in a patient is determined by immobilizing tissue proteins onto a support, derivatizing any oxidized or nitrated proteins with 2,4-dinitrophenylhydrazine (DNPH), contacting the derivatized tissue proteins with anti-DNPH antibody or anti-nitrotyrosine antibody, then measuring the amount of immunocomplex formed.

### CROSS-REFERENCE TO RELATED APPLICATION

This is a continuation-in-part of application Ser. No. 10/263,207 filed Oct. 2, 2002.

### TECHNICAL FIELD

This invention relates to assaying oxidative stress in a patient. More particularly, this invention relates to immunological methods for detecting oxidized actin and tubulin proteins, which, when present, indicate oxidative stress in a patient.

### BACKGROUND OF THE INVENTION

Inflammatory bowel disease (IBD), including ulcerative colitis (UC) and Crohn's Disease (CD), waxes and wanes between active (symptomatic) and inactive (asymptomatic) phases. The active phase of IBD is clinically manifested by the presence severe tissue damage in the bowel and intestinal barrier disruption. The level of barrier disruption correlates with the severity of IBD [Baba et al., Gut 46:830-837 (2000)].

Intestinal barrier disruption is the result of abnormal immune reactions in the mucosal tissue, which instigate an inflammatory cascade there. [Keshavarzian et al. Gastroenterology 103:177-185 (1992).] Usually, this cascade begins when the inflammatory cells, which are by-products of the abnormal immune reactions, enter into the mucosal tissue and cause the release of pro-inflammatory molecules such as reactive oxygen metabolites (e.g., superoxide anion, H₂O₂, HOCl, hydroxide radicals) and reactive nitrogen metabolites (e.g., NO) therein. [Simmonds et al. Gastroenterology 103:186-196 (1989)].

The reactive oxygen metabolites oxidize certain proteins comprised of amino acids having hydroxyl groups, resulting in the formation of carbonyl groups. Likewise, the reactive nitrogen metabolites act on proteins resulting in the formation of nitro groups. The amount of carbonyl and nitro groups present on the proteins can be measured and indicate the level of oxidative stress in the patient. Accordingly, it is critical to develop methods for evaluating levels of oxidation in tissue as a gauge of IBD disease state. Such knowledge of oxidative stress in a patient can have significant diagnostic, prognostic and therapeutic impact on this disease and others.

Carbonyl and nitro groups present on proteins can be measured using several techniques. The classic approach for the detection of protein carbonyl groups involves the reaction of the protein's carbonyl group with 2,4-dinitrophenyl-hydrazine (DNPH) followed by spectrophotometric quantification of the resulting acid hydrazones at 370 nm [Levine et al., Methods Enzymol. 233:346-357 (1994)]. Another method for carbonyl analysis is HPLC separation followed by spectroscopy at 357 nm. Carbonyl groups can also be detected by labeling with tritiated borohydride [Levine et al., Methods Enzymol. 186:464-478 (1990)].

Immunochemical techniques have been previously applied to the detection of carbonyl groups in proteins that have been purified and separated by polyacrylamide gel electrophoresis [Schacter et al., Free Radical Biol. Med. 17: 429-437 (1994); and Robinson et al., Analyt. Biochem. 266:48-57 (1999)]. A similar procedure has been used to assay proteins containing nitro groups.

### BRIEF SUMMARY OF THE INVENTION

Oxidative stress in a patient can be determined by an immunological assay for oxidized or nitrated cytoskeletal proteins in tissue. A contemplated method for assaying for the presence of oxidative stress in a patient comprises the following steps. (i) A support-bound tissue protein is provided, as by binding a predetermined amount of tissue from a patient to a solid support. (ii) The support-bound tissue protein is reacted with 2,4-dinitrophenylhydrazine (DNPH) to form a derivatized support-bound tissue protein. (iii) The derivatized support-bound tissue protein is contacted with anti-DNPH antibody and the contact is maintained for a time period sufficient to form an immunocomplex between the derivatized support-bound tissue protein and the anti-DNPH antibody. (iv) The amount of immunocomplex present is determined and compared to the amount of immunocomplex present in the same quantity of a standard sample. An amount of immunocomplex present greater than that present in the standard sample in excess of experimental error indicates the presence of oxidative stress in the patient.

A second contemplated method for assaying oxidative stress in a patient comprises the following steps. (i) A support-bound tissue protein is provided, as by binding a predetermined amount of tissue from a patient to a solid support. (ii) The support-bound tissue protein is reacted with 2,4-dinitrophenylhydrazine (DNPH) to form a derivatized support-bound tissue protein. (iii) The derivatized support-bound tissue protein is contacted with anti-nitrotyrosine antibody and the contact is maintained for a time period sufficient to form an immunocomplex between the derivatized support-bound tissue protein and the anti-nitrotyrosine antibody. (iv) The amount of immunocomplex present is determined and compared to the amount of immunocomplex present in the same quantity of a standard sample. An amount of immunocomplex present greater than that present in the standard sample in excess of experimental error indicates the presence of oxidative stress in the patient.

The present invention has several benefits and advantages. One benefit is that its use can overcome inherent problems of the prior art including the inability to analyze crude protein. An advantage of the invention is that it is relatively easy to use and is relatively inexpensive to use. Still further benefits and advantages of the invention will be apparent to those skilled in this art from the detailed description that follows.

### BRIEF DESCRIPTION OF THE DRAWINGS

In the drawings forming a part of this invention,

FIG. 1 is a graph that illustrates a slot-immunoblotting analysis of the levels of anti-nitrotyrosine (nitration) immunoreactivity of the proteins in mucosal pinch biopsies. Mucosal homogenates were analyzed by slot-blotting and processed for autoradiography and then for densitometry. Nitration immunoreactivity was expressed as follows: nitrotyrosine formation (optical density) in the patient group divided by the nitrated tissue standards, expressed as a percentage. Controls (n=10); patients with inactive UC (n=7); normal appearing, non-inflamed, mucosa of patients with active UC (n=6); inflamed, ulcerated mucosa of patients with active UC (n=15); inactive Crohn's (CC) (n=5); active Crohn's (n=6); and inflamed mucosa of patients with specific colitis (n=14). Confidence levels (P) for differences are noted as shown.

FIG. 2 is a graph that illustrates a correlation between mucosal nitrotyrosine and IBD disease severity score for patients having differing clinical disease severity scores. (Number of subjects used for analysis was as in previous figure.)

FIG. 3 is a graph that illustrates a slot-blotting analysis similar to that of FIG. 1 except for levels of carbonylation (anti-DNP) immunoreactivity of the proteins in mucosal pinch biopsies. Oxidation was expressed as carbonyl formation (i.e., optical density) in the patient group divided by the oxidized tissue standards, expressed as a percentage. From controls (n=10); patients with inactive UC (n=7); normal appearing, non-inflamed, mucosa of patients with active UC (n=6); inflamed, ulcerated mucosa of patients with active UC (n=15); inactive Crohn's (CC) (n=5); active Crohn's (n=6); and inflamed mucosa of patients with specific colitis (n=14). Confidence levels (P) for differences are noted as shown.

FIG. 4 is a graph showing the correlation between mucosal carbonylation and nitrotyrosine levels. The line is drawn by regression analysis using the parameters shown and at the confidence level shown. Number of subjects used for analysis was as above.

FIG. 5 is a graph similar to that of FIG. 2 except that mucosal carbonylation is used with IBD disease severity score. Number of subjects used for analysis was as above.

FIG. 6 is a graph showing immunoblotting analysis of anti-dinitrophenylhydrazone immunoreactivity of the actin cytoskeleton from intestinal mucosa similar to that shown in FIG. 3. Western blots of mucosal homogenates were processed for actin fractionation SDS-PAGE and then processed sequentially using monoclonal anti-DNP and HRP-conjugated-secondary antibodies. Carbonlyation of actin was expressed as carbonyl formation (i.e., optical density) in the appropriate group divided by the oxidized actin standard. Controls (n=10); patients with inactive UC (n=7); normal appearing, non-inflamed, mucosa of patients with active UC (n=6); inflamed, ulcerated mucosa of patients with active UC (n=15); inactive Crohn's (CC) (n=5); active Crohn's (n=6); and inflamed mucosa of patients with specific colitis (n=14). Confidence levels (P) for differences are noted as shown.

FIG. 7 is a graph showing immunoblotting analysis of anti-nitrotyrosine immunoreactivity of actin from the intestinal mucosa similar to FIG. 6. Western blots were processed using monoclonal anti-nitrotyrosine as the primary antibody. Representative blot from controls (n=10); patients with inactive UC (n=7); normal appearing, non-inflamed, mucosa of patients with active UC (n=6); inflamed, ulcerated mucosa of patients with active UC (n=15); inactive Crohn's (CC) (n=5); active Crohn's (n=6); and inflamed mucosa of patients with specific colitis (n=14). Confidence levels (P) for differences are noted as shown.

FIG. 8 is a graph showing quantitative immunoblotting analysis of anti-dinitrophenyl-hydrazone immunoreactivity of the tubulin cytoskeleton from mucosal biopsies similar to FIG. 6. Tubulin fractions from mucosal homogenates were separated by SDS-PAGE and analyzed by autoradiography and then by densitometry. Representatives blot from controls (n=10); patients with inactive UC (n=7); normal appearing, non-inflamed, mucosa of patients with active UC (n=6); inflamed, ulcerated mucosa of patients with active UC (n=15); inactive Crohn's (CC) (n=5); active Crohn's (n=6); and inflamed mucosa of patients with specific colitis (n=14). Confidence levels (P) for differences are noted as shown.

### DETAILED DESCRIPTION OF THE INVENTION

The present invention is directed to assaying oxidative stress in a patient. One contemplated method assays for the presence of oxidative stress in a patient and comprises the following steps: (i) A support-bound tissue protein is provided, as by binding a predetermined amount of tissue from a patient to a solid support. (ii) The support-bound tissue protein is reacted with 2,4-dinitrophenylhydrazine (DNPH) to form a derivatized support-bound tissue protein. (iii) The derivatized support-bound tissue protein is contacted with anti-DNPH antibody and the contact is maintained for a time period sufficient to form an immunocomplex between the derivatized support-bound tissue protein and the anti-DNPH antibody. (iv) The amount of immunocomplex present is determined and compared to the amount of immunocomplex present in the same quantity of a standard sample. An amount of immunocomplex present greater than that present in the standard sample in excess of experimental error indicates the presence of oxidative stress in the patient. The support-bound protein is typically washed after contact with DNPH and again after contacting with the anti-DPNH antibodies.

A second contemplated method for assaying for the presence of oxidative stress in a patient comprises the following steps: (i) A support-bound tissue protein is provided, as by binding a predetermined amount of tissue from a patient to a solid support. (ii) The support-bound tissue protein is reacted with 2,4-dinitrophenylhydrazine (DNPH) to form a derivatized support-bound tissue protein. (iii) The derivatized support-bound tissue protein is contacted with anti-nitrotyrosine antibody and the contact is maintained for a time period sufficient to form an immunocomplex between the derivatized support-bound tissue protein and the anti-nitrotyrosine antibody. (iv) The amount of immunocomplex present is determined and compared to the amount of immunocomplex present in the same quantity of a standard sample. An amount of immunocomplex present greater than that present in the standard sample in excess of experimental error indicates the presence of oxidative stress in the patient.

In a preferred embodiment, the support is a membrane such as a nitrocellulose membrane as commonly used in Western blot techniques. In another embodiment, the support is a gel matrix, such as a polyacrylamide gel for example. In yet another embodiment, the support is a porous particle similar to those found in column chromatography including HPLC such as cellulose or agarose Sephadex™, Sepharose™, and silica. In a different embodiment, the support is a plastic surface of a microtiter plate such as polystyrene or polycarbonate used in ELISA techniques. In yet another method, the support is a biological molecule such as a nucleic acid (DNA or RNA), a protein or peptide.

The amount of immunocomplex formed by the reaction of the tissue protein with DNPH is preferably determined by ultra-violet spectroscopy using the absorbance at 370 nm. In a different embodiment, the amount of immunocomplex present is determined by radiography by use of radiolabeled anti-DNPH antibodies. In yet another embodiment, the amount of immunocomplex present is determined by fluorescence spectroscopy.

In a more preferred embodiment, the amount of immunocomplex present is determined by binding a second antibody to the immunocomplex and measuring the amount of bound secondary antibody. Preferably, the second antibody is labeled with a fluorescent tag. In another embodiment, the second antibody is labeled with a radioactive molecule. In the most preferred embodiment, the second antibody is labeled with an indicator enzyme such as alkaline phosphatase or horseradish peroxidase. The amount of indicator enzyme can then be quantified by the formation of a product of an enzyme-catalyzed reaction such as by chemiluminescence techniques.

### EXAMPLE 1

### Assaying for Oxidative Stress in a Patient

Forty-seven IBD patients (20 female; 27 male; mean age=42) who underwent colonoscopic examination as part of their clinical evaluation were randomly selected. The diagnosis of IBD was established on the basis of classic clinical, endoscopic, and histological criteria. This group included 22 patients having UC, 11 having CD, and 14 with specific colitis (SC; 10 with radiation proctitis; 4 with diverticulitis). All 11 CD patients had ileo-colonic inflammation. For both UC and CD, patients were considered either active (n=15 for UC; n=6 for CD) or inactive (n=7 for UC; n=5 for CD) on the basis of disease activity indexes for UC [R. I. Breuer, et al. Gut 40, 485-91, (1997).] or CD [W. R. Best, et al. Gastroenterology 70, 439-44, (1976).]. All patients with inactive IBD were asymptomatic and had no evidence of mucosal ulceration or friability on endoscopic examination.

The majority (32 of 47) of IBD patients were taking IBD-related medications. Medications included prednisone (6 UC; 5 CD); mesalamine (13 UC; 5 CD), and the immunosuppressive medications 6-mercaptopurine or azothioprine (4 UC; 2 CD). The remaining 15 (9 UC; 6 CD) were taking no IBD-related medication. The results were compared with data obtained from ten subjects who underwent colonoscopy for evaluation of occult blood positive stool or abdominal pain, all of whom proved to have a normal colonoscopic examination (control group).

Each subject underwent colonoscopic examination after a colon preparation with Golytely solution and with conscious sedation (Versed® and Demerol®). Mucosal biopsy specimens were collected, snap frozen in liquid nitrogen and stored in a −70° C. freezer. In patients with active left-sided UC (n=6), biopsies were taken from both inflamed and non-inflamed areas of the mucosa. In patients with active CD, biopsies were only taken from grossly inflamed areas because the patchy pattern of the involvement made it difficult to accurately sample non-involved areas. This study was approved by the Institutional Review Board of Rush Presbyterian Medical Center and was performed after obtaining written consent from subjects.

Oxidation and nitration of mucosal proteins were assessed by measuring protein carbonyl and protein nitrotyrosine formation using a slot-blotting method we previously described [Robinson et al. Analytical Biochem. 266:48-57 (1999)]. The biopsied samples were homogenized and protein concentrations were assessed by the Bradford Method [Bradford, Ann Biochem. 72:224-254 (1976)].

To determine carbonyl immunoreactivity, protein samples (5 μg) were blotted to polyvinylidene difluoride (PVDF) membranes, then blocked in 5% nonfat milk in Tris-buffered saline (TBS) at room temperature for one hour. The samples were then incubated with a monoclonal rabbit anti-carbonyl antibody (1:25000, Upstate Biotech.) in blocking solution at 4° C. for overnight (about 18 hours), washed 5×for 5 minutes each wash (in 1% nonfat milk, 0.1% Tween® −20 TBS), incubated with monoclonal peroxidase-conjugated goat anti-rabbit antibody (1:5000) in blocking buffer at room temperature for one hour, and then washed again 5X. The membrane-bound proteins were soaked in enhanced chemiluminescence (ECL) reagents and exposed to ECL hyperfilm.

A similar procedure was used to determine nitrotyrosine immunoreactivity except that a monoclonal mouse anti-nitrotyrosine antibody (1:5000) was used. The relative levels of oxidized proteins were quantified by measuring the optical density (OD) of the bands corresponding to anti-carbonyl or anti-nitrotyrosine immunoreactivity with a laser densitometer. Carbonyl or nitrotyrosine formation was expressed as the ratio of carbonyl or nitrotyrosine formation in the treatment group divided by carbonyl or nitrotyrosine formation in the corresponding carbonylated or nitrated (tissue) standard run concurrently.

Oxidation and nitration of actin and tubulin were assessed by measuring carbonyl and nitrotyrosine formation as we previously described [Banan et al. J Pharmacol Exper Therap 294:997-1008 (2000).]. Briefly, tissues were homogenized and processed for PAGE fractionation and then western immunoblotting. Identity of bands was confirmed as actin or tubulin by comparison with standards run concurrently. In separate blots, specific monoclonal anti-actin or anti-tubulin antibody further confirmed the identity of actin or tubulin. To avoid oxidation during sample processing, all buffers contained 0.5 mM dithiothreitol (DTT) and 20 mM 4,5-dihydroxy-1,3-benzene sulfonic acid (Sigma, St. Louis, Mo.).

Samples were blotted to a PVDF membrane followed by successive incubations in 2 N HCl and 2,4-dinitrophenylhydrazine (DNPH, 100 μg/ml in 2 N HCl; Sigma, St. Louis, Mo.) for 5 minutes each. Membranes were washed 3×in 2 N HCl and then washed 7×in 100% methanol (5 minutes each), followed by blocking for 1 hour in 5% BSA in 10×PBS/Tween 20 (PBS-T). Membranes were then incubated for 1 hour in 1% BSA/PBS-T buffer containing anti-DNPH [1:25000 dilution] (Molecular Probes, Eugene, Oreg.) and further incubated with an HRP-conjugated secondary antibody [1:4000 dilution, 1 hour] (Molecular Probes, Eugene, Oreg.).

To determine the nitrotyrosine content of actin or tubulin a similar method was used except following the blocking step above (i.e., BSA/PBS-T buffer), membranes were incubated with 2 pg/ml monoclonal anti-nitrotyrosine antibody for 1 hour (Upstate Biotech., Lake Placid, N.Y.) followed by the HRP-conjugated secondary antibody as above. Wash steps and film exposure were as in a standard western blot protocol. Relative levels of oxidized or nitrated actin or tubulin were then quantified by measuring, with a laser densitometer, the OD of the bands corresponding to anti-DNP or anti-nitrotyrosine immunoreactivity. Comparing OD values, immunoreactivity was expressed as the percentage of carbonyl or nitrotyrosine formation in the treatment group compared to that in the maximally oxidized or nitrated actin or tubulin standard, run concurrently.

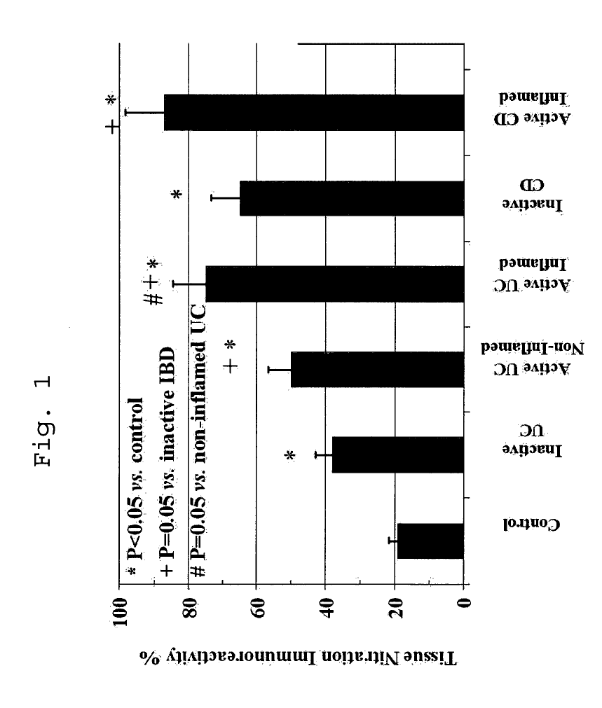

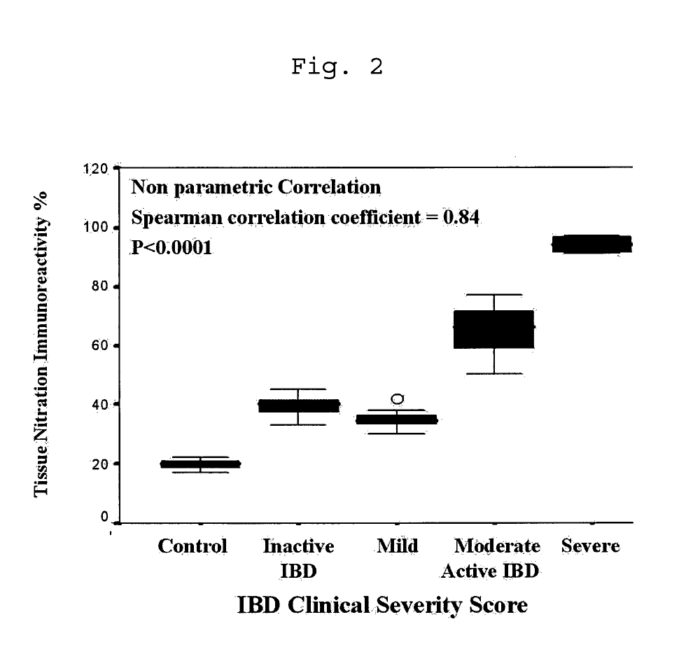

The results show that mucosal nitration (nitrotyrosine immunoreactivity) was elevated in patients with IBD regardless of disease type or disease activity (FIG. 1) with (a) active IBD (UC and CD) being higher than inactive IBD, (b) active IBD higher than active Specific Colitis, and (c) active CD similar to active UC. Nitrotyrosine levels in normal-appearing mucosa from patients with active left-sided UC were significantly higher than inflamed mucosa from the same patients (paired analysis). There were positive correlations between nitrotyrosine and disease severity score (FIG. 2).

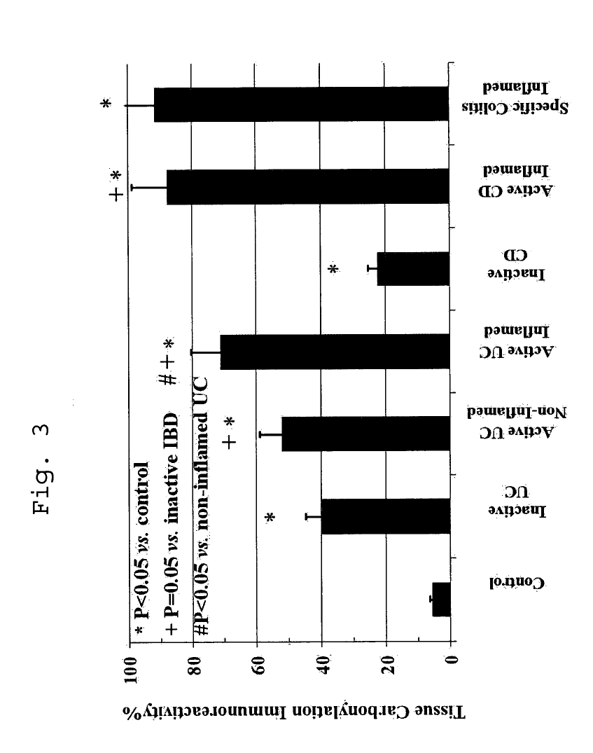

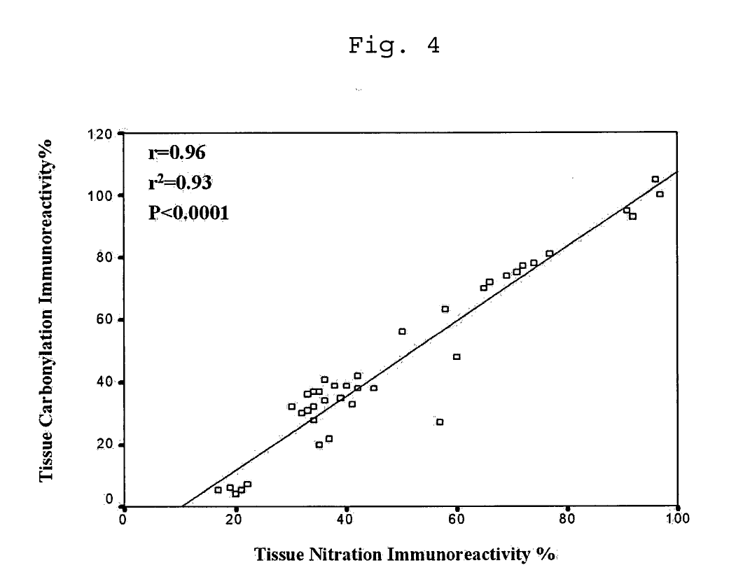

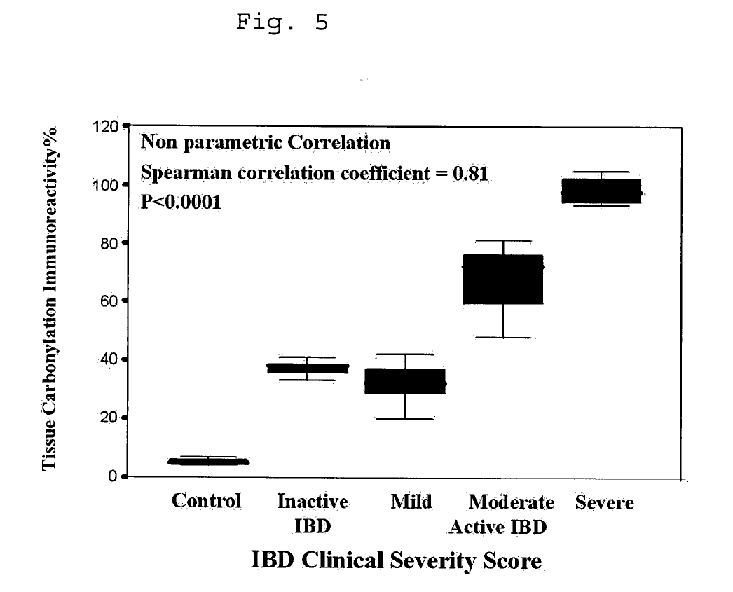

Likewise, carbonylation was higher in all patients with IBD regardless of disease type (FIG. 3). Carbonylation was higher in active CD and UC than inactive CD and UC, respectively. Carbonylation was higher in inflamed mucosa than in non-inflamed mucosa of the same patients with active UC (paired analysis). There were no significant differences between carbonyl levels in the inflamed mucosa of patients with active UC compared to active CD or active specific colitis. Similar to nitration, positive correlations were observed between carbonylation and nitrotyrosine levels (FIG. 4), and carbonylation and disease severity score (FIG. 5).

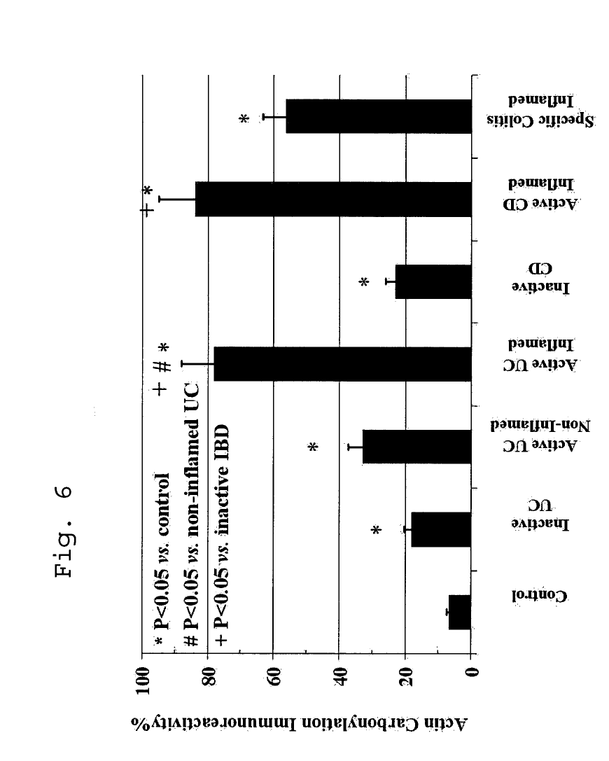

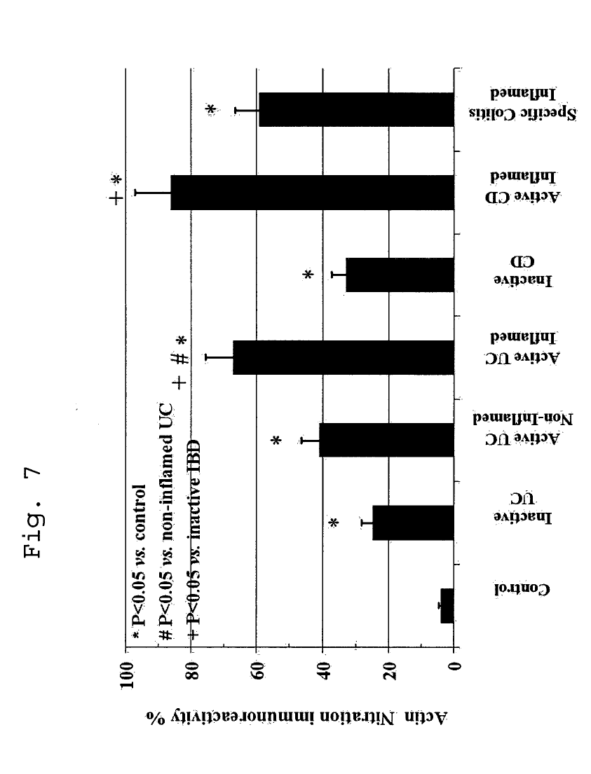

Moreover, both carbonylation (FIG. 6) and nitration (FIG. 7) of actin (43 kDa) were increased in the mucosa of patients with colitis regardless of type or activity. Actin nitration and carbonylation were significantly less in the non-inflamed mucosa of patients with active UC than in inflamed mucosa from the same patient (paired analysis). Both actin nitration and carbonylation were higher in active CD and UC than the corresponding inactive disease.

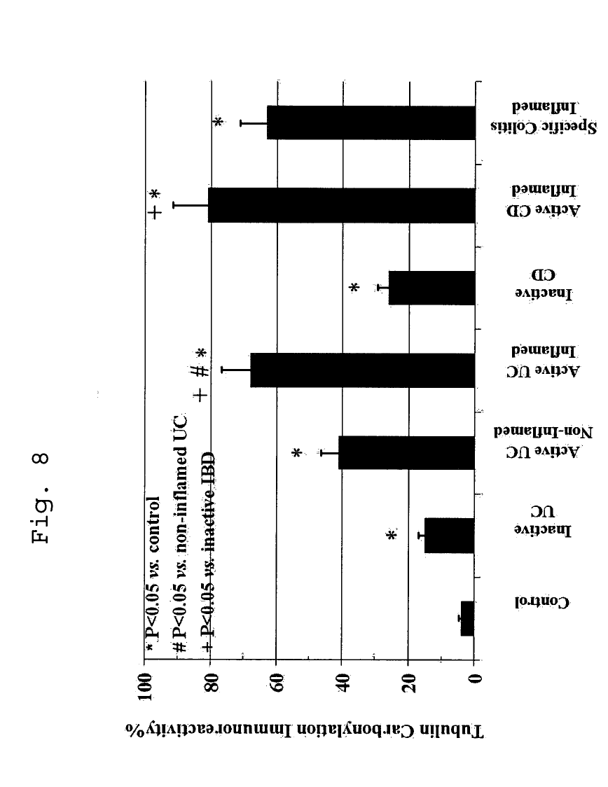

Similar to actin, tubulin (50 kDa) was carbonylated more in the colonic mucosa of patients with colitis (FIG. 8). Carbonyl levels were less in inactive disease than in inflamed mucosa of active disease. Tubulin carbonylation in non-inflamed mucosa was significantly less than in inflamed mucosa in the same active UC patient (paired analysis). There was no significant difference between tubulin carbonylation and actin carbonylation.

From the foregoing, it will be observed that numerous modifications and variations can be effected without departing from the true spirit and scope of the present invention. It is to be understood that no limitation with respect to the specific examples presented is intended or should be inferred. The disclosure is intended to cover by the appended claims modifications as fall within the scope of the claims. Each of the patents and articles cited herein is incorporated by reference. The use of the article “a” or “an” is intended to include one or more.

What is claimed:

1. A method assaying for the presence of oxidative stress in a patient comprising the steps of:
(i) providing a predetermined amount of protein from a patient bound to a support forming a support-bound tissue protein;
(ii) reacting the support-bound tissue protein with 2,4-dinitrophenylhydrazine (DNPH) to form a derivatized support-bound tissue protein;
(iii) contacting the derivatized support-bound tissue protein with anti-DNPH antibody and maintaining that contact for a time period sufficient to form an immunocomplex between the derivatized support-bound tissue protein and the anti-DNPH antibody; and
(iv) determining the amount of immunocomplex present and comparing that amount to the amount of immunocomplex present in the same quantity of a standard sample, the amount greater than that present in the standard sample in excess of experimental error indicating the presence of oxidative stress in the patient.

2. The method of claim 1 wherein the support is a membrane.

3. The method of claim 1 wherein the support is a gel matrix.

4. The method of claim 1 wherein the support is a porous particle.

5. The method of claim 1 wherein the support is a plastic surface.

6. The method of claim 1 wherein the support is a biological molecule.

7. The method of claim 1 wherein the support is a nucleic acid.

8. The method of claim 1 wherein the support is a protein.

9. The method of claim 1 wherein the amount of immunocomplex present is determined by ultra-violet spectroscopy.

10. The method of claim 1 wherein the amount of immunocomplex present is determined by radiography.

11. The method of claim 1 wherein the amount of immunocomplex present is determined by fluorescence spectroscopy.

12. The method of claim 1 wherein the amount of immunocomplex present is determined by binding the immunocomplex to a second antibody and measuring the amount of bound secondary antibody.

13. The method of claim 12 wherein the second antibody is labeled with a fluorescent tag.

14. The method of claim 12 wherein the second antibody is labeled with a radioactive molecule.

15. The method if claim 12 wherein the second antibody is labeled with an indicator enzyme.

16. A method assaying for the presence of oxidative stress in a patient comprising the steps of:
(i) providing a predetermined amount of protein from a patient bound to a support forming a support-bound tissue protein;
(ii) reacting the membrane-bound tissue protein with 2, 4, dinitrophenylhydrazine (DNPH) to form a derivatized membrane-bound tissue protein;
(iii) contacting the derivatized membrane-bound tissue protein with anti-DNPH antibody and maintaining that contact for a time period sufficient to form an immunocomplex between the derivatized membrane-bound tissue protein and the anti-DNPH antibody; and
(iv) determining the amount of immunocomplex formed wherein the amount formed is determined by binding the immunocomplex to a second antibody that is labeled with horseradish peroxidase and measuring the amount of bound secondary antibody labeled with horseradish peroxidase and comparing that amount to the amount of bound secondary antibody labeled with horseradish peroxidase present in the same quantity of a standard sample, the amount greater than that present in the standard sample in excess of experimental error indicating the presence of oxidative stress in the patient.

17. A method assaying for the presence of oxidative stress in a patient comprising the steps of:
(i) providing a predetermined amount of protein from a patient bound to a support forming a support-bound tissue protein;
(ii) reacting the support-bound tissue protein with 2,4-dinitrophenylhydrazine (DNPH) to form a derivatized support-bound tissue protein;
(iii) contacting the derivatized support-bound tissue protein with anti-nitrotyrosine antibody and maintaining that contact for a time period sufficient to form an immunocomplex between the derivatized support-bound tissue protein and the anti-nirtotyrosine antibody; and
(iv) determining the amount of immunocomplex present and comparing that amount to the amount of immunocomplex present in the same quantity of a standard sample, the amount greater than that present in the standard sample in excess of experimental error indicating the presence of oxidative stress in the patient.

18. The method of claim 17 wherein the support is a membrane.

19. The method of claim 17 wherein the support is a gel matrix.

20. The method of claim 17 wherein the support is a porous particle.

21. The method of claim 17 wherein the support is a plastic surface.

22. The method of claim 17 wherein the support is a biological molecule.

23. The method of claim 17 wherein the support is a nucleic acid.

24. The method of claim 17 wherein the support is a protein.

25. The method of claim 17 wherein the amount of immunocomplex present is determined by ultra-violet spectroscopy.

26. The method of claim 17 wherein the amount of immunocomplex present is determined by radiography.

27. The method of claim 17 wherein the amount of immunocomplex present is determined by fluorescence spectroscopy.

28. The method of claim 17 wherein the amount of immunocomplex present is determined by binding the immunocomplex to a second antibody and measuring the amount of bound secondary antibody.

29. The method of claim 28 wherein the second antibody is labeled with a fluorescent tag.

30. The method of claim 28 wherein the second antibody is labeled with a radioactive molecule.

31. The method if claim 28 wherein the second antibody is labeled with an indicator enzyme.

32. A method assaying for the presence of oxidative stress in a patient comprising the steps of:
(i) providing a predetermined amount of protein from a patient bound to a support forming a support-bound tissue protein;
(ii) reacting the membrane-bound tissue protein with 2, 4, dinitrophenylhydrazine (DNPH) to form a derivatized membrane-bound tissue protein;
(iii) contacting the derivatized membrane-bound tissue protein with anti-nitrotyrosine antibody and maintaining that contact for a time period sufficient to form an immunocomplex between the derivatized membrane-bound tissue protein and the anti-nitrotyrosine antibody; and
(iv) determining the amount of immunocomplex formed wherein the amount formed is determined by binding the immunocomplex to a second antibody that is labeled with horseradish peroxidase and measuring the amount of bound secondary antibody labeled with horseradish peroxidase and comparing that amount to the amount of bound secondary antibody labeled with horseradish peroxidase present in the same quantity of a standard sample, the amount greater than that present in the standard sample in excess of experimental error indicating the presence of oxidative stress in the patient.
